(19) United States
(12) Patent Application Publication
(10) Pub. No.: US 2005/0032051 A1
(43) Pub. Date: Feb. 10, 2005

(54) Rapid flow-based immunoassay microchip

(76) Inventors: Mark A Hayes, Tempe, AZ (US); Nolan A. Polson, Longmont, CO (US); Anil K. Vuppu, Tempe, AZ (US); Timothy A. Crowley, Tempe, AZ (US); Antonio A. Garcia, Tempe, AZ (US)

Correspondence Address: Pitney Hardin LLP, 685 Third Avenue, New York, NY, 10017-4024, US

(21) Appl. No.: 10/296,489

(22) PCT Filed: Jun. 19, 2001
(86) PCT No.: PCT/US01/41037

Related U.S. Application Data
(60) Provisional application No. 60/212,255, filed on Jun. 19, 2000.
(60) Provisional application No. 60/221,798, filed on Jul. 31, 2000.

(51) Int. Cl. C12Q 1/68; C12M 1/34

(52) U.S. Cl. 435/6; 435/287.2

(57) ABSTRACT

A microimmunoassay arrangement including paramagnetic particles having labeled antibody held in a microchannel by an external magnetic field provides rapid analysis using small sample volumes.

### SPECIFICATION

This invention relates to a microimmunoassay arrangement including paramagnetic particles in a microchannel. The paramagnetic particles contain antibodies which can be used to detect the presence of an antigen. An external magnetic field is used to reversibly arrange the paramagnetic particles in the microchannel.

### BACKGROUND OF THE INVENTION

Immunoassays are one of the most important analytical methods used today in clinical laboratories.

Conventional, non-microchip based automated immunoassay techniques detect biological materials in the low attomole concentration range, give results in 20 to 30 minutes, have critical variance (“CV”) based on concentration of 5 to 10% and are low cost to produce ($0.35/test).

The drive towards Point of Care (POC) technology has produced several rapid immunoassay devices that yield results at a doctor's office or clinic within minutes. POC technology continues to strive to lower the unit cost of a test, produce better patient outcomes through early diagnosis, and increase sensitivity and accuracy.

Existing rapid inmmunoassay devices utilize a wide variety of techniques for detection and antibody immobilization. Unfortunately, nearly all of the devices available are yes/no tests with no quantitation. Typically, these assays and test strips including solid phase immunochemistry.

Medical laboratory researchers rely principally on microplate immunoassays. The assays take 2 hours to run and requires 100-200 microliters of sample. In certain applications such as surgery or rapid diagnosis in trauma or acute conditions, fast analysis is of very high importance. Standard immunoassay tests can take 2 hours or more even if the highly technical equipment and a trained individual are readily available to run the test. More specifically the overriding need in cancer surgeries is to provide a rapid test that will help the decision making process and aid in surgical removal of all cancerous tissue since immunological markers are present at high concentrations mitigating the need for highly sensitive assays.

There are several aspects to POC immunoassays which are crucial to improving test accuracy, sensitivity and lowering costs that are not currently addressed. It would be useful to concurrently run multiple tests using a single sample. Another important aspect is reducing the risk of exposure to infectious fluids by reducing sample size. Also, more forward thinking on immunodiagnostics is to some day monitor hormonal changes in real-time or nearly real-time.

Conventional clinical immunoassays require long incubation times, extensive wash procedures, and relatively large quantities of costly reagents. By switching to small particles as the stationary phase, much larger surface areas are obtained for possible binding sites, creating a much more sensitive and higher throughput immunoassay. To overcome these drawbacks, investigations to the feasibility of performing immunoassays in micro-channels have been performed with success. These studies have demonstrated both enzyme and competitive immunoassays on a microchip format where binding constants and kinetic constants (Kₘ, Kᵢ) have been determined.

While these studies have shown the ability to perform competitive immunoassays on a microchip, none have demonstrated the ability to perform a sandwich type assay. Typically these types of assays are considered heterogeneous due to the solid support structure needed for primary antibody immobilization. In a sandwich assay, a primary antibody is immobilized onto the solid phase. Both the antigen and tag antibody are introduced in liquid form sequentially separated by wash steps. In a sandwich assay, no further separation of free form versus complexed antigen-antibody is needed as the solid phase is immobilized. For this type of assay to be performed in microchannels, the solid support beads must be immobilized by either physical or magnetic barriers.

An immunoassay which uses solid phase immobilization has been demonstrated in microchannels but has been limited to polystyrene bead substrates. In this assay antigen was immobilized onto polystyrene beads followed by a tagged antibody complexation. Although this study did indicate the potential of reducing assay time by performing clinical immunoassays on a microchannel format, there are some inherent limitations to this system. The immobilization of polystyrene beads requires the use of restriction barriers which adds to the complexity of the fabrication process. Further, a system which is easily fabricated which would allow for the rapid regeneration or exchange of the solid support is desired.

Accordingly, there is a need for an immunoassay which can give results quickly, uses small sample volumes, is compact in size, is flexible and is economical.

### SUMMARY OF THE INVENTION

An object of the invention is to provide a microimmunoassay arrangement which can provide for rapid analysis using small sample volumes.

Another object of the invention is to provide a microimmunoassay arrangement which can be used for multiple antigen analyses.

A further object of the invention is to provide a microimmunassay arrangement which is reusuable.

These and other objects of the invention are achieved by providing a microimmunoassay arrangement comprising a plurality of channels. A plurality of paramagnetic particles located in at least one of the channels includes immobilized antibodies which have a label attached thereto. The paramagnetic particles are held in place by a magnetic gradient.

The invention also provides for a method of determining the presence of an antigen in a sample comprising providing a plurality of paramagnetic particles wherein the magnetic particles comprise an antibody including a label, applying an external magnetic field to the microchannel, introducing the sample into the microchannel and detecting the presence of an antigen in the sample.

### BRIEF DESCRIPTION OF THE DRAWINGS

Further objects and advantages of the present invention will be more fully appreciated from a reading of the detailed description when considered with the accompanying drawing wherein:

FIG. 1 is a schematic of a microimmunoassay arrangement in accordance with the claimed invention;

FIG. 2 is a top view schematic of a microchip arrangement in accordance with the invention;

FIG. 3 is a graph of fluorescence versus time for reaction of anti-FITC with FITC in an arrangement in accordance with the invention;

FIG. 4 is a graph of fluorescence versus time during regeneration of a bed of paramagnetic particles including FITC in an arrangement according to the invention;

FIG. 5 is a graph of fluorescence versus concentration for a bed of regenerated paramagnetic particles including FITC in an arrangement according to the invention; and

FIG. 6 is a graph of fluorescence versus concentration for a newly packed bed of paramagnetic particles including FITC in an arrangement according to the invention.

### DETAILED DESCRIPTION OF THE PREFERRED EMBODIMENTS

The present invention provides a microimmunassay arrangement in which paramagnetic particles including an immobilized antibody are constrained in a microchannel using an externally applied magnetic field.

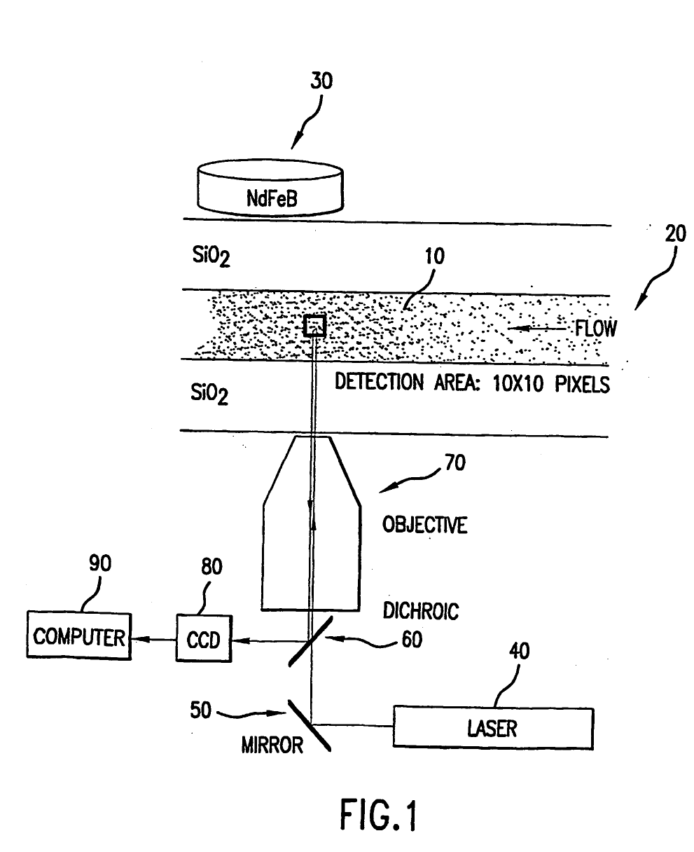

An embodiment of a microimmunoassay arrangement in accordance with the invention is shown in FIG. 1. Paramagnetic particles 10 are constrained in a microchannel 20 by an external magnet 30. Antibodies including a fluorescent label are immobilized on the paramagnetic particles 10. A laser 40 emits light which is reflected by mirror 58 and transmitted to a dichroic filter 50 through a microscopic objective 70 and onto the particles 10. The fluorescent light emitted by the paramagnetic particles is detected by a CCD camera 80 and the data is transmitted to a computer 90.

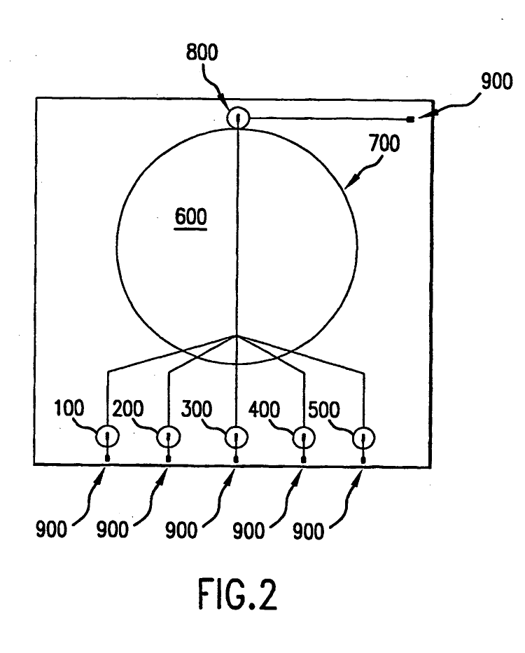

A schematic of a top view of a microchip (700) in accordance with the invention is shown in FIG. 2. Circles labeled (100-500) are ports for the microbore capillary and reservoirs of wash buffers, immunochemicals, and imaging reagents. The ports (100-500) are connected to the microchannel (600) containing the paramagnetic particles. A rare earth magnet (not shown) which holds the paramagnetic particles in the channel is placed below the microchip (700). Fluid exits the microchip through the exit port (800). Electroosmosis propels the fluid through energization of microelectrode leads (900).

The invention will be better understood from, but is not limited to the Examples below. The reagents and materials referred to in the Examples are as follows. Sodium dihydrogen phosphate (NaH₂PO₄) and fluorescein isotyiocyanate (FITC) were obtained from Aldrich Chemical Company, Inc. (Milwaukee, Wis.) and were used as received. Biotinylated monoclonal anti-FITC; glutaraldehyde, and thiourea were obtained from Sigma Chemical Company (St. Louis, Mo.) and used as received. All NaH₂PO₄ buffers were prepared to a 20 mM concentration and adjusted to pH 7.2 using 1M sodium hydroxide (Mallincrodt, Phillipsburg, N.J.). Amino paramagnetic beads (1-2 μm diameter) were purchased from Polysciences, Inc. (Warrington, Pa.) and were used as received. Dynal streptavidin labeled paramagnetic particles, 2.8 μm diameter, 1 mg/mL, was obtained from Nichols Institute Diagnostics (San Juan Capistrano, Calif.). All buffers and samples were degassed under vacuum for 5 minutes and were filtered with a Millex-LCR Filter Unit 0.5 μm pore size (Bedford, Mass.). All buffers and samples were prepared with 18 M purified water drawn from a NANOpure UV ultrapure water filtration system (Bamnstead, Dubuque, Iowa).

Paramagnetic beads were labeled with monoclonal-anti-FITC using two methods. One method involves the use of silver linked paramagnetic beads reacted with biotylated anti-FITC to form covalent bonds as described in Boner, M. R. Dissertation Arizona State University, Tempe 1999 and Ramirez-Vick, J., Lee, J. Garcia, A. A., Reactive and Functional Polymers, 2000, 43, 53-62. The other method uses a streptavidin-biotin binding mechanism. Biotinylated monoclonal anti-FITC (30 μL) was incubated with the streptavidin labeled paramagnetic beads (100 μL) for 1 hour prior to use where the anti-FITC antibody to streptavidin-biotin binding site ratio was maintained at 2:1.

Experiments were performed in 20 μm microchannels in fused silica capillaries (150 μm outer diameter (o.d.)/50 μm inner diameter (i.d.)) which were purchased from Polymicro Technologies (Phoenix, Ariz.) and cut to 50.8 cm in length.

An Olympus IX70 Inverted Research microscope (Tokyo, Japan) was used for imaging. An Omnichrome Model 100 HeCd laser was used as the fluorescence excitation source (442 nm). Image acquisition in the packed bed areas was performed with an RS170 CCD camera (CSI Electronics, East Hartford, Conn.) integrated with National Instruments LabVIEW IMAQ image acquisition software and hardware (National Instruments, Austin, Tex.). Data analysis on the images was done by averaging a 10×10 pixel area (5.7 μm×5.7 μm) in the middle of the microchannels followed by further data processing using MathCAD 7.0 (MathSoft, Inc., Cambridge, Mass.) and Excel (Microsoft Corporation, Seattle, Wash.) programs.

### EXAMPLE 1

Vacuum induced pressures provided the fluidic transport within this experiment. The paramagnetic beads were typically packed under 0.33 atm vacuum for 30 seconds, followed by a 0.101 atm vacuum for 2 minutes. This provided a uniformly packed bed of approximately 1-2 mm in length (given 50.8 cm capillary length, 50 μm inner diameter). Once introduced into the microchannels, the paramagnetic beads were locally restrained by the application of a magnetic field. A rare earth magnet used to generate the external field is a ¾″ diameter, 0.1875″ thick disk of NdFeB (27/30 mixed), rated at 11 lbs. lift (Edmund Scientific, Barrington, N.J.; Cat. Number: CR35-106). Previously labeled anti-FITC paramagnetic beads were packed for a total bed length of 1-2 mm. Fluorescence was monitored before FITC introduction into the microchannel, during the FITC interaction, and after a buffer wash following FITC exposure using laser induced fluorescence. The optical train for this experimental procedure required no special alignment or cross beam techniques. Fluorescence intensity was simply observed through microscope objectives and analysis was accomplished on the resulting images via the CCD camera.

Flushing of the packed bed was accomplished easily by removal of the magnetic field, such that the beads were swept away in the bulk buffer flow. Based upon the flow rates of the system, the packed bed could be flushed out almost immediately and a subsequent bed could be packed as a replacement after use. This type of packing is more advantageous over similar studies using polystyrene beads in microchannels because there is no need for extensive fabrication procedures. The beds can be generated and flushed out quickly allowing for higher through-put heterogeneous immunoassays in microchannels. Additionally, if array-like channels are used, the induced magnetic field can pack beds of paramagnetic beads equally well in arrays of channels as it can in a single channel.

### EXAMPLE 2

To validate the microimmunoassay system, initial control experiments were done to discount any increase in fluorescence due to non-specific binding. Primary antibody was loaded onto the 1-2 μm diameter paramagnetic beads. A 360 μm o.d./50 μm i.d. fused silica capillary (50.8 cm length) was used for this experiment. The beads were introduced into the capillaries by applying 0.33 atm vacuum to a counter reservoir for 30 seconds followed by 0.101 atm vacuum for 2 minutes. Buffer was then flushed through the system for 10 minutes to allow for system equilibrium. A neodymium-iron-boron (NdFeB) magnet was placed over the capillary prior to introduction of the beads to ensure the formation of a packed bed. Two initial experiments were performed, one using paramagnetic beads labeled with monoclonal anti-FITC antibody, and the other with non-labeled paramagnetic beads. After the bed was packed 1 mM FITC was flushed through the bed for 15 minutes followed by a buffer rinse to remove free FITC from the detection area. Fluorescence intensity was recorded immediately after the FITC was washed from the detection zone (t=0 min) and at t=30 minutes. In the experiment where non-labeled paramagnetic beads were used, immediately after the FITC plug exited the detection window the fluorescence intensity dropped to background levels and remained there for full 30 minutes. The anti-FITC labeled paramagnetic beads showed an increase in residual fluorescence signal. Immediately after the FITC plug exited the detection window, the residual fluorescence was 15.5±3.1 and leveled off at 8.9±0.5 above background levels after the 30 minutes of washing. This shows that build-up of fluorescence is primarily due to anti-FITC/FITC interactions and not due to non-specific binding of FITC onto the paramagnetic beads themselves.

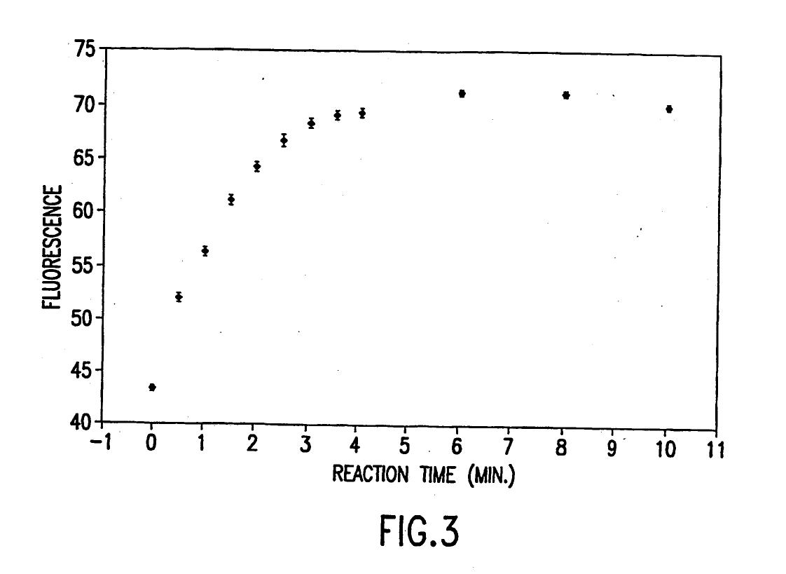

Subsequent experiments were performed to determine optimal reaction time, sensitivity, and stability. A new packed bed of anti-FITC labeled paramagnetic beads (2.8 μm diameter beads, 2 mm bed length) was formed for each experiment using the streptavidin-biotin procedure. The paramagnetic beads were introduced into the fused silica capillary using the same conditions as described previously. Buffer was flushed through the packed bed for 40 minutes to allow for stabilization of the system. A plug of 125 μm FITC was introduced into the microchannel using 0.101 atm vacuum at sequential time intervals (0 to 4 minutes; step=30 seconds; 4 to 10 minutes; step=2 minutes) followed by a standard 4 minute wash and imaged for 10 seconds as shown in FIG. 3. The maximum signal obtained in this experiment was 70.4±0.9. This shows that the optimal reaction time for the microimmunoassay (FITC/anti-FITC) is achieved after 3 minutes (90% of maximum signal) of bed exposure to the FITC molecule. This is much quicker than both conventional microtiter plate systems and polystyrene microchannel systems. Typically conventional microtiter plate FITC immunoassays require a reaction time of 45 to 120 minutes. The drastic improvement in the system comes from two primary sources, maximum diffusion distance/times and surface area to volume ratios. In this experiment, the entire bed volume is <4 pL where paramagnetic beads are <1 μm apart. The calculated diffusion constant (D) for FITC is 3.5×10⁻⁶ cm²/s so the time that it would require for a FITC molecule in solution to diffuse across 1 μm is ˜300 milliseconds. In conventional microtiter plate immunoassays the diffusion distances are 1.3 to 2.3 mm where diffusion times required for the anti-FITC/FITC complexation are 2700 to 7200 seconds. This result in a much greater mass transport mechanism in the flow through immunoasssay system as opposed to the microtiter plate system. The surface area to volume ratio relates how large the reaction field (available binding sites) is as compared to the volume of solution passing through the bed. The surface area to volume ratio for the paramagnetic microimmunoassay experiments presented here is approximately 85,000. This represents a 6500 fold increase over conventional microtiter plate assays (S/V=13 cm⁻¹) and a 180 fold increase over competing polystyrene immunoassays in microchannels (S/V=480 cm⁻¹).

### EXAMPLE 3

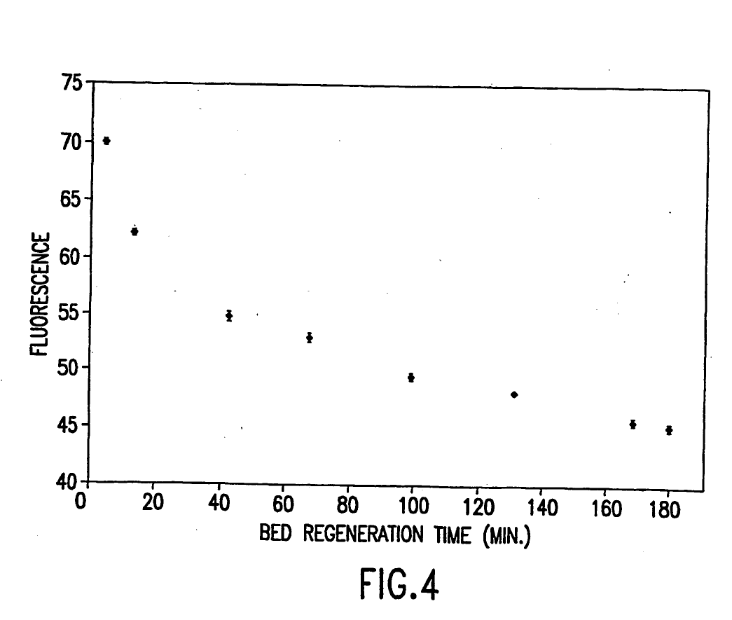

A bed regeneration experiment was performed following Example 2. In this experiment, buffer was washed over the paramagnetic beads labeled with anti-FITC (same bed conditions as reaction time studies) after 125 μm FITC was exposed to the bed for 10 minutes where laser induced fluorescence was monitored over time as shown in FIG. 4. Approximately one hour after the buffer wash was initiated, the packed bed fluorescence has decreased to within 35% of background levels. After approximately two hours, the fluorescence has decreased to within 16%, and after three hours, the fluorescence signal is within 5% of background fluorescence levels.

### EXAMPLE 4

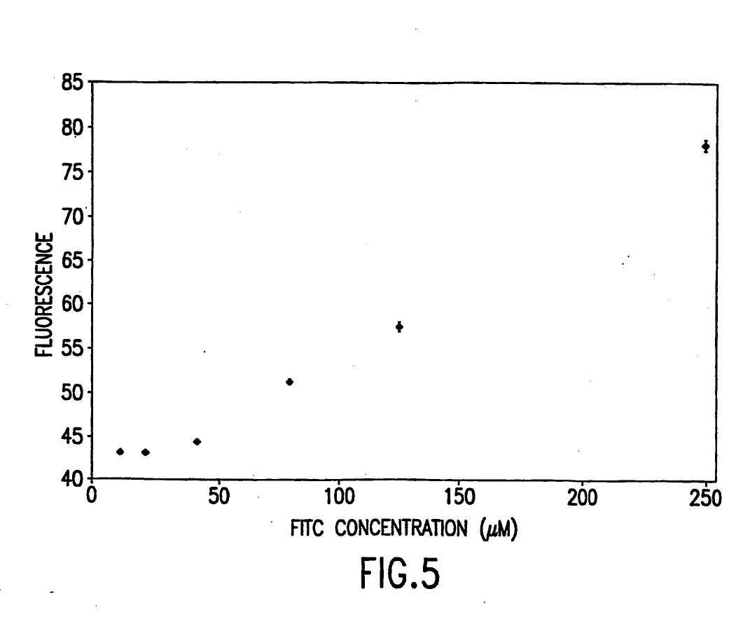

In order to determine if the anti-FITC antibodies on the paramagnetic beads were still active after regeneration, various concentrations of FITC were passed through the bed to verify if anti-FITC/FITC complexation occurred as shown in FIG. 5. FITC was exposed to the paramagnetic beads using a 0.101 atm vacuum for 3.5 minutes. Buffer was flushed through the system for 4 minutes and monitored for laser induced fluorescence. For this regenerated bed, the limits of detection were approximately 40 μM with a linear curve fit of y=0.151x+39.623 (R²=0.992). This shows that the anti-FITC labeled paramagnetic beads can be regenerated after use for further processing.

### EXAMPLE 5

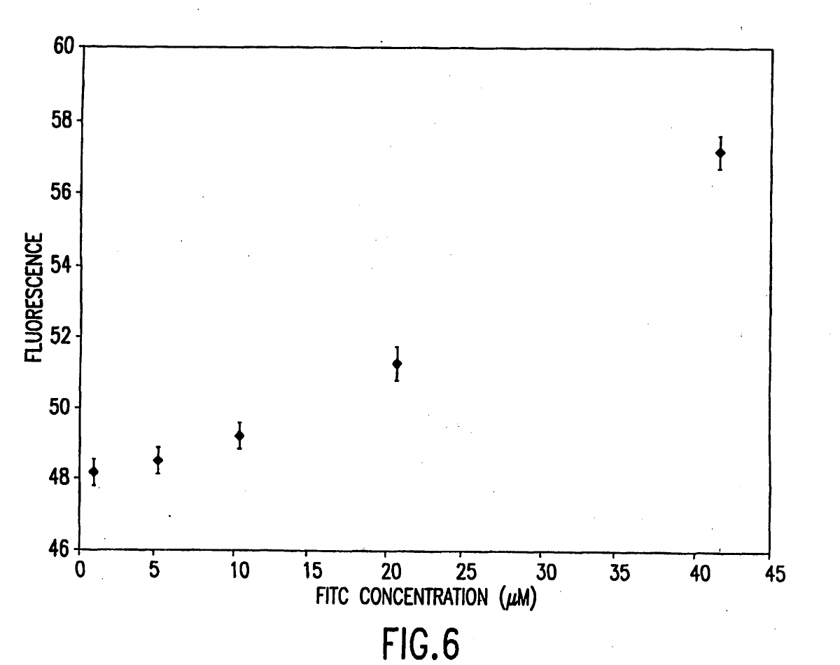

Experiments were performed on a newly packed paramagnetic bed to determine the optimal detection limits and sensitivity of the system. In order to verify if the anti-FITC effected the FITC quantum efficiency the following analysis was done. A first mixture of FITC to anti-FITC (1:1 molar ratio ˜20 pmoles) and a second mixture of FITC diluted to the same concentration as the first (405 nM FITC) were flowed through an unpacked microchannel. Laser induced fluorescence was recorded and compared. The fluorescence intensity of the diluted FITC sample was 6.35±0.32 over background levels. For the anti-FITC/FITC solution, the fluorescence intensity was 2.31±0.31 over background levels. This indicates that the FITC/anti-FITC complexation has a 64% quenching effect on the fluorescence signal. This has been observed in similar systems. Due to the quenching effect the detection limit of the system will be higher. However, in similar systems where no quenching takes place, i.e. sandwich assays, more sensitive measurements can be experimentally observed. A newly packed bed of streptavidin-biotin anti-FITC labeled paramagnetic beads (2.8 μm diameter beads, 2 mm bed length) was used for the calibration curve to verify concentration detection limits. Buffer was flushed through the system for 3 hours to ensure a stable background fluorescence signal. Various concentration of FITC were passed through the bed region (0.101 atm vacuum) for 4 minutes followed by a 4 minute buffer wash as illustrated in FIG. 6. This data represents a linear curve fit according to y=0.226x+47.249 (R²=0.974). Based on the background fluorescence signal, the relative detection limit in this case was approximately 10 μm which is above detection limits observed for laser induced fluorescence (sub-nM detection limits). However, the mass detection limit for the microimmunoassay is more significant. In these studies, the detection area was defined as 10×10 pixels (5.7 μm×5.7 μm) where the laser penetration depth was assumed to be 2 μm. For the Dynal paramagnetic beads used in this study, the calculated number of binding sites per bead is 3.9×10⁶. Given a void volume in the packed bed of 40%, the actual number of beads within the detection area is approximately 3.5. If half of the surface area of each bead is probed for detection, there are only 1.3×10⁷ binding sites within the detection zone. If all sites are occupied within the detection area, mass detection limits are 22 attomoles. To further decrease the detection of limits for subsequent systems, antigens where the flourophore does not take part in the actual binding process (minimized quenching effects) should be used.

The experiments demonstrate the feasibility of using small diameter paramagnetic beads for use in heterogeneous immunoassay applications in microchannels. Labeled paramagnetic beads are easily packed in a microchannel in the presence of a strong magnetic field. Removal and regeneration of the packed bed was accomplished quickly without complicated procedures. The optimal reaction time for the anti-FITC/FITC complexation occurred at 3 minutes where 90% of the maximum fluorescent signal was obtained. The anti-FITC labeled paramagnetic beads could be regenerated to within 5% of background levels. The regenerated bed was sensitive down to approximately 40 μM FITC. A newly packed bed of the same material was sensitive down to 10 μM FITC. The detection limits were higher than expected due to the quenching effect (64%) of the anti-FITC antibody on the FITC molecule. The present invention provides ultrafast analysis times for capture and associated washing steps; integration of magnetic immobilization for ease of use and economy; reuse of microchip in solid phase immunoassays; flexibility for any immunoassay and multiple antigen analysis using a single chip; direct detection of particles for fast analysis; good sensitivity through judicious choice of optics and signal processing and allows the use of ultrasmall volumes thereby minimizing exposure to hazardous fluids, and for use in small animal medical research and forensics.

1. A microimmunassay arrangement comprising
a microchip comprising a plurality of channels,
a plurality of paramagnetic particles located in at least one of the channels, the paramagnetic particles comprising an antibody including a label,
a magnet located external to the at least one channel, and
a detector.

2. A microimmunoassay arrangement according to claim 1, wherein the label is a fluorescent label and the detector is a fluorescence detector.

3. A microimmunoassay arrangement according to claim 2, further comprising a laser.

4. A microimmunoassay arrangement according to claim 1, wherein the at least one channel comprises fused silica.

5. A method of determining the presence of an antigen in a sample comprising:
providing a plurality of magnetic particles in a microchannel, wherein the magnetic particles comprise an antibody including a label,
applying an external magnetic field to the microchannel,
introducing the sample into the microchannel, and
detecting the presence of an antigen in the sample.

6. A method according to claim 5, wherein the label is a fluorescent label.

7. A method according to claim 6 comprising detecting the presence of an antigen in the sample by detecting a change in fluorescence.
